(19) United States
(12) Patent Application Publication
(10) Pub. No.: US 2020/0361084 A1
(43) Pub. Date: Nov. 19, 2020

(54) SIGNAL PROCESSING DEVICE AND SIGNAL PROCESSING METHOD, FORCE DETECTION DEVICE, AND ROBOT DEVICE

(71) Applicant: SONY CORPORATION, Tokyo (JP)

(72) Inventor: KAZUO HONGO, CHIBA (JP)

(21) Appl. No.: 16/638,516

(22) PCT Filed: Jul. 20, 2018
(86) PCT No.: PCT/JP2018/027254

§ 371 Date: Feb. 12, 2020

(30) Foreign Application Priority Data
Aug. 21, 2017 (JP) 2017-158947

(51) Int. Cl. B25J 9/16; B25J 15/00; B25J 13/08; G01L 1/22; A61B 34/30

(52) U.S. Cl. CPC B25J 9/1633; B25J 15/0019; B25J 9/06; G01L 1/22; A61B 34/30; B25J 13/085

(57) ABSTRACT

A signal processing device for processing a detection signal of a sensor is provided. The signal processing device branches a detection signal of a sensor into a plurality of paths, and performs different preprocessing before AD conversion for each of the paths to generate a plurality of detection signals. For example, a first path for performing AD conversion of a signal of a first sensitivity, the signal being obtained by amplifying the detection signal of the sensor to match the first sensitivity, and a second path for attenuating the signal of the first sensitivity and performing AD conversion of a signal of a second sensitivity lower than the first sensitivity, are included, and the detection signals having different sensitivities are generated. Alternatively, an offset of the signal of the first sensitivity is changed for each of the paths, and a plurality of detection signals having different measurement ranges is generated.

### TECHNICAL FIELD

The technology disclosed in the present specification relates to a signal processing device and a signal processing method for processing a detection signal of a sensor, a force detection device that detects a force on the basis of a detection signal of a sensor attached to a strain generation body, and a robot device that measures an external force applied to an end effector.

### BACKGROUND ART

Recent advances in robot technology are remarkable, and force sensors are used for various purposes. Examples of the various purposes include a purpose of performing collaborative work with humans, a purpose of performing actions that depend on the shape of the object, such as tracing, a purpose of using it as a criterion for making the robot learn, a purpose of ensuring the quality as log data for work, and the like.

Generally, a force sensor is configured such that a pair of strain detection sensors is attached to facing sides of a strain generation body. Therefore, six or more pairs of strain sensors are used for a six-axis force sensor. Then, when measuring six-axis forces, matrix calculation is performed for signals obtained from the six pairs of strain sensors, so that the signals are converted into the six-axis forces (specifically, translational forces in X, Y, and Z-axes directions and torques around the respective axes).

A correlation arising from the structure of the strain generation body to be used is inevitably caused between a translational force and a torque measured using a force sensor. For example, in a case of using a force sensor attached to a proximal end side of a robot hand, a ratio of the translational force to the torque to be measured significantly varies depending on the length of the hand, the mass of an object gripped by the hand, or the like, and thus sometimes significantly differs from a ratio of a translational force to a torque of the structure of the selected strain generation body. Meanwhile, there is a limit to the lineup of force sensors that can be actually prepared. This is because it is difficult to produce a strain generation body having appropriate size and mass with a desired ratio of the translational force to the torque within a machinable shape and within a practical price.

For example, a force sensor has been proposed, in which an inner member and an outer member are connected by a plurality of arc-shaped arms having a property of causing elastic deformation at least in part (for example, see Patent Document 1). When an external force acts on the inner member in a state where the outer member is fixed, elastic deformation occurs in an arc-shaped arm, and displacement occurs in the inner member. Therefore, the force sensor is configured to electrically detect the elastic deformation of the arc-shaped arm by a detection element to detect the applied external force.

### CITATION LIST

### Patent Document

Patent Document 1: Japanese Patent Application Laid-Open No. 2016-70709

### SUMMARY OF THE INVENTION

### Problems to be Solved by the Invention

An object of the technology disclosed in the present specification is to provide a signal processing device and a signal processing method for adaptively processing a detection signal of a sensor in an appropriate measurement range with an appropriate sensitivity, a force detection device that adaptively processes a detection signal of a sensor attached to a strain generation body in an appropriate measurement range with an appropriate sensitivity to detect a force, and a robot device that measures an external force applied to an end effector.

### Solutions to Problems

The technology disclosed in the present specification has been made in consideration of the above-described problems, and the first aspect of the technology is a signal processing device including a signal processing unit configured to branch a detection signal of a sensor into a plurality of paths, and perform different preprocessing before AD conversion for each of the paths to generate a plurality of detection signals.

The signal processing device according to the first aspect includes a first path for performing AD conversion of a signal of a first sensitivity, the signal being obtained by amplifying the detection signal of the sensor to match the first sensitivity, and a second path for attenuating the signal of the first sensitivity and performing AD conversion of a signal of a second sensitivity lower than the first sensitivity, and generates the plurality of detection signals having different sensitivities. Alternatively, the signal processing device according to the first aspect includes a path for changing an offset of a signal of a first sensitivity, the signal being obtained by amplifying the detection signal of the sensor to match the first sensitivity, and performing AD conversion.

Furthermore, the second aspect of the technology disclosed in the present specification is a signal processing method including a signal processing step of branching a detection signal of a sensor into a plurality of paths, and performing different preprocessing before AD conversion for each of the paths to generate a plurality of detection signals.

Furthermore, the third aspect of the technology disclosed in the present specification is a force detection device including a signal processing unit configured to branch a detection signal of a sensor attached to a strain generation body into a plurality of paths, and perform different preprocessing before AD conversion for each of the paths to generate a plurality of detection signals.

Furthermore, the fourth aspect of the technology disclosed in the present specification is:

a robot device including:

an end effector;

a force sensor attached to a proximal end side of the end effector; and

a signal processing unit configured to process a detection signal of the force sensor, in which

the force sensor includes a strain generation body and a sensor that detects deformation of the strain generation body, and

the signal processing unit branches the detection signal of the sensor, and performs different preprocessing before AD conversion for each path to generate a plurality of detection signals. The end effector may include a medical instrument.

### Effects of the Invention

According to the technology disclosed in the specification, a signal processing device and a signal processing method for adaptively processing a detection signal of a sensor in an appropriate measurement range with an appropriate sensitivity, a force detection device that adaptively processes a detection signal of a sensor attached to a strain generation body in an appropriate measurement range with an appropriate sensitivity to detect a force, and a robot device that measures an external force applied to an end effector can be provided.

Note that the effects described in the present specification are merely examples, and the effects of the present invention are not limited thereto. Furthermore, the present invention may further exhibit additional effects in addition to the above effects.

Still other objects, features, and advantages of the technology disclosed in the present specification will become clear from detailed description based on embodiments described later and attached drawings.

### BRIEF DESCRIPTION OF DRAWINGS

FIG. 1 is a view illustrating a configuration example of a six-axis force sensor 100.

FIG. 2 is a view illustrating a configuration example of a forceps 200 having a force sensor 201 arranged on a proximal end side.

FIG. 3 is a diagram illustrating a configuration example of a signal processing circuit 300 that processes a detection signal of a strain sensor.

FIG. 4 is a diagram illustrating a modification of the signal processing circuit 300.

FIG. 5 is a diagram illustrating a state in which a measurement range is shared by N-multiplexed amplifiers.

FIG. 6 is a diagram illustrating another example in which a measurement range is shared by N-multiplexed amplifiers.

FIG. 7 is a diagram illustrating another modification of the signal processing circuit 300.

FIG. 8 is a view illustrating a configuration example of a robot arm 800 to which a force sensor 801 is attached.

FIG. 9 is a diagram illustrating an operation example of the signal processing circuit 300 illustrated in FIG. 3.

FIG. 10 is a diagram illustrating an operation example of the signal processing circuit 300 illustrated in FIG. 4.

FIG. 11 is a diagram illustrating an operation example of the signal processing circuit 300 illustrated in FIG. 4.

FIG. 12 is a diagram illustrating a configuration example of a signal processing circuit 1200 according to a second example.

FIG. 13 is a diagram illustrating an operation example of the signal processing circuit 1200.

FIG. 14 is a diagram illustrating an operation example of the signal processing circuit 1200.

FIG. 15 is a diagram illustrating an operation example of the signal processing circuit 1200.

FIG. 16 is a diagram illustrating a configuration example of a signal processing circuit 1600 according to the second example.

FIG. 17 is a diagram illustrating an operation example of the signal processing circuit 1600.

FIG. 18 is a diagram illustrating an operation example of the signal processing circuit 1600.

FIG. 19 is a diagram illustrating an operation example of the signal processing circuit 1600.

FIG. 20 is a diagram illustrating an operation example of the signal processing circuit 1600.

FIG. 21 is a diagram illustrating a configuration example of a signal processing circuit 2100 according to the second example.

FIG. 22 is a diagram illustrating a configuration example of a signal processing circuit 2200 according to the second example.

### MODE FOR CARRYING OUT THE INVENTION

Hereinafter, embodiments of the technology disclosed in the present specification will be described in detail with reference to the drawings.

As a technique of detecting a force, there is typically a technique of attaching a strain detection sensor (hereinafter simply referred to as “strain sensor”) to a strain generation body having a structure that is easily locally deformed when a force is applied and converting a deformation amount of the strain generation body measured by the strain sensor into a force level.

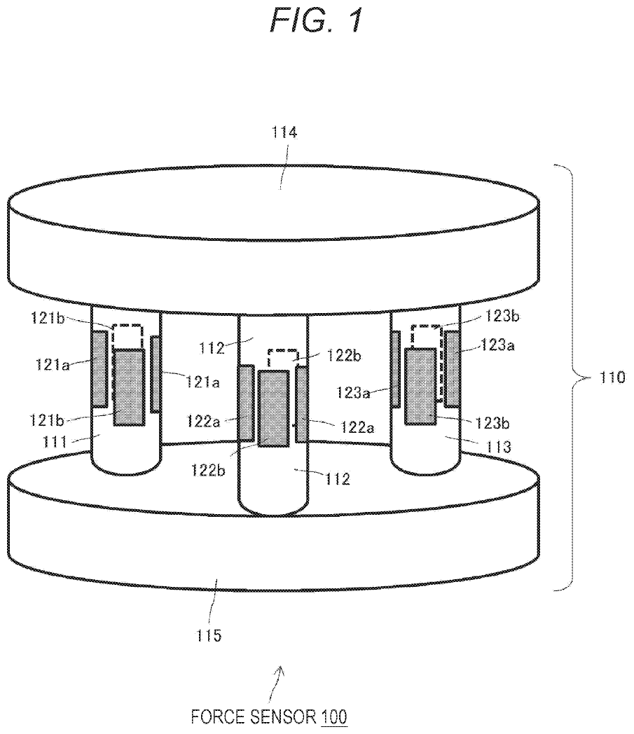

FIG. 1 illustrates a configuration example of a six-axis force sensor 100 including a strain generation body 110 and strain sensors 121, 122, and 123.

The strain generation body 110 includes a top plate 114 and a bottom plate 115 having relatively high rigidity, and three elongated supports 111, 112, and 113 supporting the top plate 114 on the bottom plate 115. Examples of the material of the strain generation body 110 include nickel chrome molybdenum steel, stainless steel, aluminum alloy, and the like. The supports 111, 112, and 113 are flexible, and strain sensors 121a and 121b, 122a and 122b, and 123a and 123b are attached to side surfaces, respectively. Note that each of the strain sensors 121a and 121b, 122a and 122b, and 123a and 123b includes a set of strain sensor elements arranged to face each other. Note that the reason why two facing strain sensors are used as one set is to cancel a component caused by temperature change to compensate for the temperature, and is known as a two-gauge method.

Note that detection elements of various types such as piezoelectric type, magnetic type, optical type, and capacitance type, in addition to strain gauges, can be applied to the strain sensors 121a and 121b, 122a and 122b, and 123a and 123b.

For example, when an external force in an arbitrary direction is applied between the top plate 114 and the bottom plate 115, at least one of the support 111, 112, or 113 is deformed such as being compressed, extended, or bent. The strain sensors 121a and 121b, 122a and 122b, and 123a and 123b are integrally deformed with the corresponding supports 111, 112, and 113, respectively. For example, in the case of a strain gauge-type strain sensor, an electrical resistance changes according to the deformation amount. The change in electrical resistance can be captured as a change in voltage in an arithmetic device (not illustrated), for example, and can be converted into a force level. Then, matrix calculation is performed using a predetermined calibration matrix for the three sets of strain sensors 121a and 121b, 122a and 122b, and 123a and 123b, so that six-axis forces and rotational torques can be measured.

Since signals output from the strain sensors 121, 122, and 123 are analog signals, the signals are converted into digital signals of N bits (where N is a positive integer) by an AD converter and then taken into an arithmetic device such as a personal computer or a robot control device, and used for calculation such as conversion to a force level. Here, in a case where the analog signal output by the strain sensor is converted into a 10-bit digital signal by AD conversion, for example, a measurable rating for a minimum resolution is the 10th power of 2, that is, 1024. Therefore, in a case where the strain sensors 121, 122, and 123 are deformed to 1024 times or more of the minimum resolution, a maximum value cannot be acquired. That is, the deformation amount exceeding the rating is unknown.

In the case of the force sensor 100 having the degree of freedom in a plurality of axes as illustrated in FIG. 1, forces applied to the axes are applied to each of the strain sensors 121a and 121b, 122a and 122b, and 123a and 123b in a complex manner. Therefore, a relationship among force detection sensitivities actually measurable in the plurality of axes is determined from the structure of the strain generation body 110 or the like.

A correlation arising from the structure of the strain generation body to be used is inevitably caused between a translational force and a torque measured using a force sensor. For example, in a case of using a force sensor attached to a proximal end side of a robot hand, a ratio of the translational force to the torque to be measured significantly varies depending on the length of the hand, the mass of an object gripped by the hand, or the like, and thus sometimes significantly differs from a ratio of a translational force to a torque of the structure of the selected strain generation body.

However, there is a limit to the lineup of force sensors that can be actually prepared. This is because it is difficult to produce a strain generation body having appropriate size and mass with a desired ratio of the translational force to the torque within a machinable shape and within a practical price.

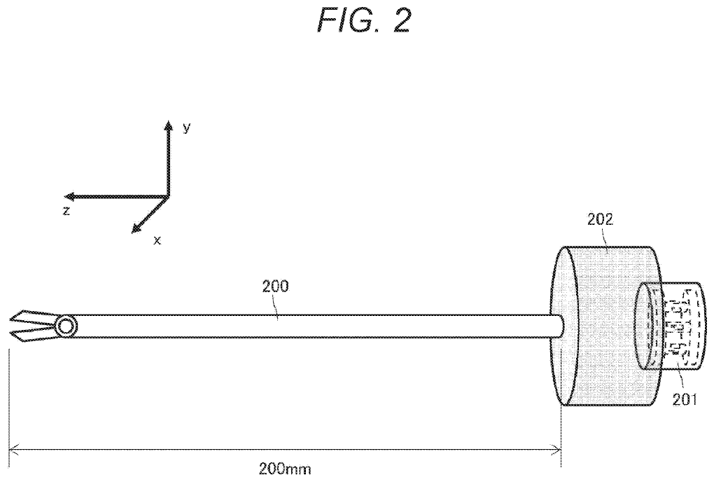

For example, in a medical robot used in a surgical operation, a case of arranging a force sensor 201 on a proximal end side of a medical forceps 200, as illustrated in FIG. 2, for the purpose of measuring a force applied to a distal end of the forceps as an end effector, will be considered. Note that the length from the distal end of the forceps 200 to the force sensor 201 is 200 mm. The force sensor 201 is assumed to have a six-axis configuration as illustrated in FIG. 1, for example. Furthermore, in the illustrated example, the force sensor 201 is attached to a rear stage of a drive unit 202 for the forceps 200.

In a case where a force of 1 N is applied to the distal end of the forceps 200 in x, y, and z directions, translational forces of Fₓ=1 N, F_{y}=1 N, and F_{z}=1 N act on the x, y, and z directions, and moments of Mₓ=200 Nmm, M_{y}=200 Nmm, and M_{z}=0 Nmm act around the axes. Considering the weight of the forceps 200, the rating of the force sensor 201 at this time requires about Fₓ=10 N, F_{y}=10 N, and F_{z}=10 N and Mₓ=500 Nmm, M_{y}=500 Nmm, and M_{z}=100 Nmm. That is, the ratio of the translational force to the torque is large and is extremely unbalanced because the forceps 200 is long and the distance from the vicinity of the distal end of the forceps 200 to which an external force is applied to the force sensor 201 is relatively long.

It is difficult to design and produce a force sensor in which the translational force and torque to be measured are unbalanced. This is because it is difficult to produce a strain generation body having appropriate size and mass with a desired ratio of the translational force to the torque within a machinable shape and within a practical price.

Furthermore, in a case where the ratio of the translational force to the torque significantly varies depending on the use even if the ratio of the translational force to the torque falls within a developable range of the strain generation body, the structure of the strain generation body needs to be re-examined in accordance with the variation.

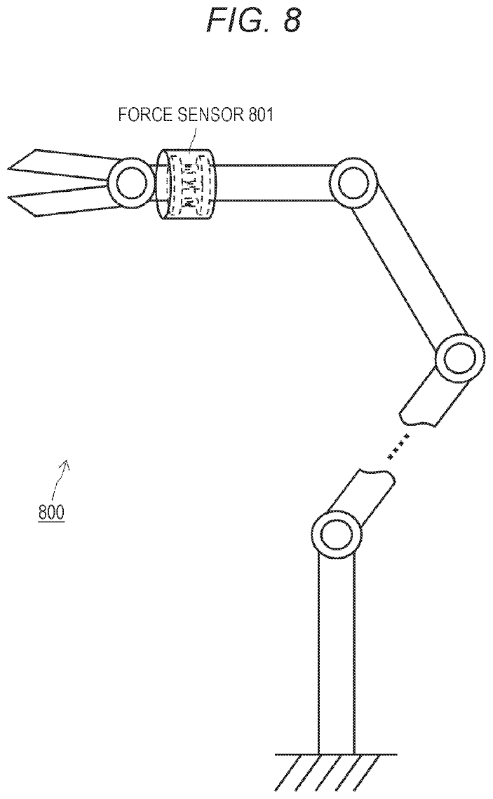

For example, in a case of mounting a force sensor 801 on a wrist portion of a robot arm 800, as illustrated in FIG. 8, the distance from an application point of an external force to the force sensor is relatively short, so the ratio of the translational force to the torque is relatively well balanced, unlike the example illustrated in FIG. 2.

To cope with the unbalance of the ratio of the translational force to the torque by the mechanical structure of the strain generation body, the structure of the strain generation body needs to be changed every time a detection balance between desired translational force and torque changes. For this reason, a product including many kinds of strain generation bodies needs to be prepared, which is a disadvantage in mass production. Furthermore, the detection balance between the translational force and the torque that can be achieved by a single strain generation body structure is in a narrow range, and it is easy to fall into a structure limit.

Furthermore, an electrical solution that enables measurement in a wide range from a minute signal to an excessive signal using automatic gain control or a polygonal line gain circuit without depending on the mechanical structure of the strain generation body is also conceivable. However, there is no implementation example in a method of electrically adjusting the detection balance between the translational force and the torque.

Therefore, the present specification will hereinafter propose a technology of branching an output signal of a sensor such as a strain sensor, and multiplexing an amplifier and generating a plurality of signals having different amplification factors to simultaneously create signals having different sensitivities at different rating levels, thereby coping with a wide range of change in an output level of the sensor. By applying this technology to processing for the output signal of the strain sensor, the wide range of change in the ratio of the translational force to the torque can be coped with without changing the structure of the strain generation body. In other words, since there is no feedback or the like, there is an advantage of generating no delay for a use requiring a high speed. Furthermore, the present technology can also be used for processing for an output signal of a potentiometer, in addition to the force sensor.

### FIRST EXAMPLE

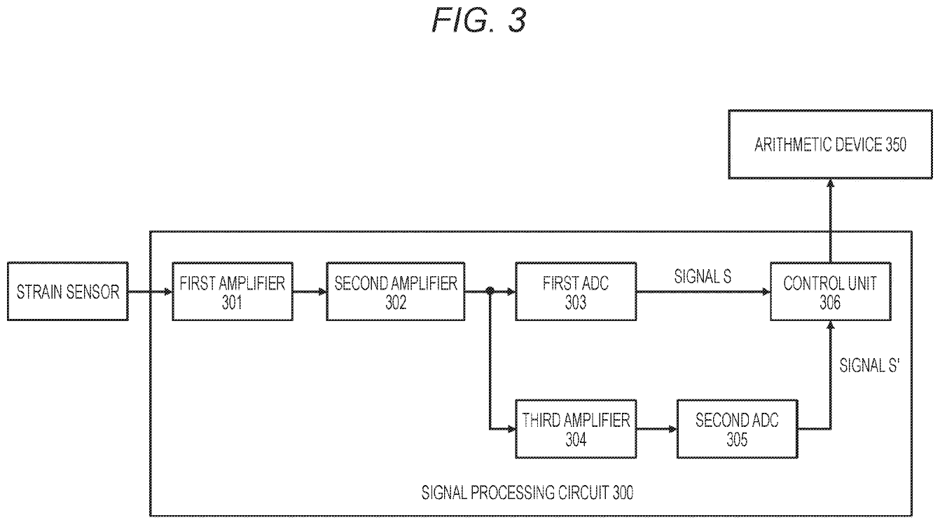

FIG. 3 illustrates a configuration example of a signal processing circuit 300 that processes a detection signal of a strain sensor according to a first example. For example, the illustrated signal processing circuit 300 is implemented in the form of an amplifier device connected to the force sensor 100, a communication unit that transmits an output signal of the force sensor 100 to an arithmetic device 350 such as a personal computer or a robot control device, or the like.

The strain sensor in the figure corresponds to one of the strain sensors 121a and 121b, 122a and 122b, and 123a and 123b in the force sensor 100 illustrated in FIG. 1, for example. Basically, it is to be understood that the signal processing circuit 300 as illustrated in FIG. 3 is provided for each of a pair of strain sensors, and detection signals of the strain sensors are processed. Furthermore, it is to be understood that the signal processing circuit 300 can be applied to processing for a detection signal of another sensor such as a potentiometer instead of a strain sensor.

Translational forces and torques applied to a plurality of axes are applied to each of the strain sensors 121a and 121b, 122a and 122b, and 123a and 123b in a complex manner. Therefore, the detection signal of each of the strain sensors 121a and 121b, 122a and 122b, and 123a and 123b includes a plurality of components of the translational forces and torques. In the case where the unbalance between the translational force and the torque to be measured is assumed, as described above, there is a demand of desirably detecting the translational force F_{z} in the z-axis direction with a high sensitivity but desirably detecting the translational forces and torques in the other axial directions with a low sensitivity to reduce the influence of noise, for example. Furthermore, when an overload is applied to an object to be measured or the object to be measured moves at a high speed, a high resolution is not necessary and detection with a low sensitivity is sufficient. Therefore, a detection signal of a single strain sensor needs to be amplified in accordance with a plurality of sensitivities.

A first amplifier 301 receives the detection signal of the strain sensor and amplifies the detection signal with low noise. Furthermore, a second amplifier 302 amplifies the detection signal after low-noise amplification with a predetermined amplification factor, and further appropriately performs processing such as offset adjustment as necessary. It is to be understood that the detection signal of the strain sensor is amplified to achieve a necessary (or high) sensitivity that meets the purpose by two-stage amplification processing using the first amplifier 301 and the second amplifier 302.

An output signal of the second amplifier 302 is branched into two paths having different amplification factors. In one of the paths, the output signal of the second amplifier 302 is directly converted into a digital signal by a first AD converter (ADC) 303 and input to a control unit 306 at a subsequent stage as a high-sensitivity detection signal S. That is, in the one of the paths, the detection signal S corresponding to F_{z} to be detected with a high sensitivity is created although a measurement range is narrow.

Furthermore, in the other path, the output signal of the second amplifier 302 is further amplified by a third amplifier 304, converted into a digital signal by a second AD converter 305, and input to the control unit 306 at a subsequent stage. Specifically, the third amplifier 304 is an attenuator that attenuates an input signal to 1/n (where n>1), and the input signal is converted into a digital signal by the second AD converter 305 and is then input to the control unit 306 as a low-sensitivity detection signal S′.

That is, in the other path, the detection signal S′ corresponding to the axial translational force and torque other than F_{z} to be detected with a low sensitivity with reduced noise influence is created over a wide measurement range. For example, the third amplifier 304 weakens (or attenuates) the detection signal S such that the resolution becomes about ¼ of the maximum value of values that can be taken by the high-sensitivity detection signal S to reduce the influence of noise or breaking strength in which the strain generation body and the strain sensor do not break falls in the measurement range. Note that the third amplifier 304 may be a variable amplifier having a variable attenuation factor (1/n).

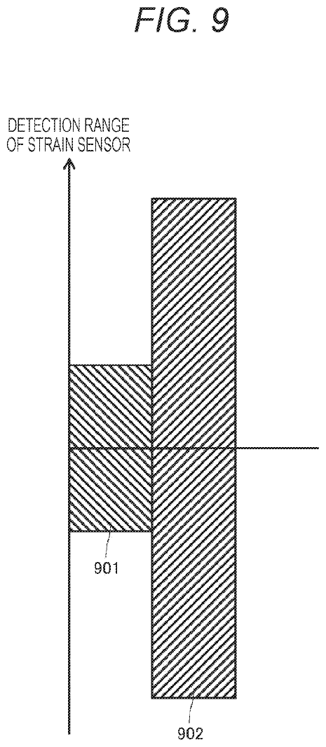

FIG. 9 illustrates respective measurement ranges of the detection signal S output from the first AD converter 303 and the detection signal S′ output from the second AD converter 305 in the signal processing circuit 300 illustrated in FIG. 3. Although a measurement range 901 of the detection signal S is narrow, the strain of the strain sensor can be measured with a high resolution. Meanwhile, a measurement range 902 of the detection signal S′ is wide, and the strain of the strain sensor can be measured even in a region beyond the measurement range 901 of the detection signal S. The wide measurement range 902 of the detection signal S′ is due to suppression of the detection signal S by the second AD converter 305. Although the influence of noise can be reduced, the resolution is low. Therefore, the signal processing circuit 300 has a detection range corresponding to the measurement range 902, but the resolution decreases in a region outside the detection range corresponding to the measurement range 901.

Then, the control unit 306 performs communication of digital data of the plurality of detection signals S and S′ having different sensitivities obtained from one strain sensor to the external arithmetic device (personal computer or robot control device) 350 and other types of digital processing.

As described above, according to the signal processing circuit 300 illustrated in FIG. 3, signals having different sensitivities at different rating levels can be simultaneously created by branching the detection signal of the strain sensor and generating the plurality of detection signals having different amplification factors. Therefore, the external arithmetic device side that receives signals from the signal processing circuit 300 performs calculation of converting a signal into a force using either the detection signal having the high sensitivity in the narrow measurement range or the detection signal having the low sensitivity in the wide measurement range as necessary, of the detection signals of the strain sensors, for each axis, thereby coping with the wide range of change in the ratio of the translational force to the torque. For example, the signal processing circuit 300 can meet the demand of desirably detecting the translational force F_{z} in the z-axis direction with a high sensitivity but desirably detecting the other translational forces and torques in the other axial directions with a low sensitivity to reduce the influence of noise on the arithmetic device 350 side.

Furthermore, according to the configuration of the signal processing circuit 300 illustrated in FIG. 3, there is no feedback or the like, and thus there is an advantage of generating no delay for a use requiring a high speed.

In FIG. 3, the signal processing circuit 300 and the arithmetic device 350 have been described focusing on the detection signals of one set of strain sensors. In the case of the six-axis force sensor 100 as illustrated in FIG. 1, a total of six sets of the strain sensors 121a and 121b, 122a and 122b, and 123a and 123b are provided. Therefore, the above signal processing circuit 300 is provided for each of the strain sensors. Here, six high-sensitivity signals (S₁, S₂, S₃, S₄, S₅, and S₆) and six low-sensitivity signals (S₁′, S′₂, S′₃, S′₄, S′₅, and S′₆) with a suppressed sensitivity are supplied to the arithmetic device 350, where a high-sensitivity signal is Si and a low-sensitivity signal is Si′, which are simultaneously created from the detection signal of the i-th strain sensor by the signal processing circuit 300.

Then, the arithmetic device 350 can calculate the high-sensitivity six-axis translational forces and torques Fₓ, F_{y}, Fₓ, Mₓ, M_{y}, and M_{z} from the high-sensitivity signals (S₁, S₂, S₃, S₄, S₅, and S₆) by matrix calculation using a predetermined calibration matrix, as illustrated in the following expression (1).

$$\begin{matrix}[ Math . 1 ] & \\ ( \begin{matrix}{F}_{x} \\ ⋮ \\ {M}_{z}\end{matrix} ) = ( \begin{matrix}{K}_{11} & … & {K}_{16} \\ ⋮ & ⋱ & ⋮ \\ {K}_{61} & … & {K}_{66}\end{matrix} ) ( \begin{matrix}{S}_{1} \\ ⋮ \\ {S}_{6}\end{matrix} ) & ( 1 )\end{matrix}$$

Furthermore, the arithmetic device 350 can calculate the low-sensitivity six-axis translational forces and torques Fₓ′, F_{y}′, F_{z}′, Mₓ′, M_{y}′, and M_{z}′ from the low-sensitivity signals (S₁′, S′₂, S′₃, S′₄, S′₅, and S′₆) by matrix calculation using a predetermined calibration matrix, as illustrated in the following expression (2).

$$\begin{matrix}[ Math . 2 ] & \\ ( \begin{matrix}{F}_{x}^{′} \\ ⋮ \\ {M}_{z}^{′}\end{matrix} ) = ( \begin{matrix}{K}_{11}^{′} & … & {K}_{16}^{′} \\ ⋮ & ⋱ & ⋮ \\ {K}_{61}^{′} & … & {K}_{66}^{′}\end{matrix} ) ( \begin{matrix}{S}_{1}^{′} \\ ⋮ \\ {S}_{6}^{′}\end{matrix} ) & ( 2 )\end{matrix}$$

The arithmetic device 350 side can calculate the translational force or the torque separately using the high-sensitivity signal Sᵢ and the low-sensitivity signal Sᵢ′ for each axis. Furthermore, when one of high-sensitivity signals Sᵢ′ has reached an upper limit, the arithmetic device 350 side can supplement calculation partially using the low-sensitivity signal Sᵢ′ (where i is an integer of 1 to 6). A signal can be acquired with a high sensitivity in the range where a signal can be measured with the high-sensitivity signal Sᵢ. Meanwhile, in the range of the low-sensitivity signal Sᵢ′, the sensitivity is greatly inferior to that in the case of using Sᵢ, but an unrated signal that cannot be conventionally measured can be acquired. Specifically, F_{z} is measured with a high sensitivity, and the other axes can be used even in a state where the sensitivity is suppressed. The maximum measurement range can be said to be extended while maintaining the high resolution of the force sensor 100.

For example, even in the case where the ratio of the translational force to the torque is large and is extremely unbalanced, as illustrated in FIG. 2, the force sensor 100 can be used as it is without changing the structure of the strain generation body 100. Specifically, the translational force F_{z} is input as it is to the force sensor 201 but the translational forces in the other directions act as F_{y} and F_{z} and also act as moments M_{y} and M_{z} according to the length of a moment arm, where a longitudinal direction of the forceps 200 is the z-axis direction.

Normally, since the forceps 200 is inserted into a body through a small hole (an abdominal cavity, a chest cavity, or the like) using a trocar and used and thus the moment arm is inevitably long, the moments M_{y} and M_{z} are detected as larger values than the translational force F. Therefore, when measuring the moments M_{y} and M_{z}, the force sensor 201 has better balance when the sensitivity is suppressed.

According to the present embodiment, the moments M_{y} and M_{z} can be measured using the low-sensitivity detection signal while measuring the translational force F_{z} using the high-sensitivity detection signal as described in the above expressions (1) and (2) without changing the strain generation body structure of the force sensor 201. Therefore, even in a case where the force sensor 201 is applied to the forceps 200 that is long in the longitudinal direction, the force sensor 201 can be sufficiently balanced.

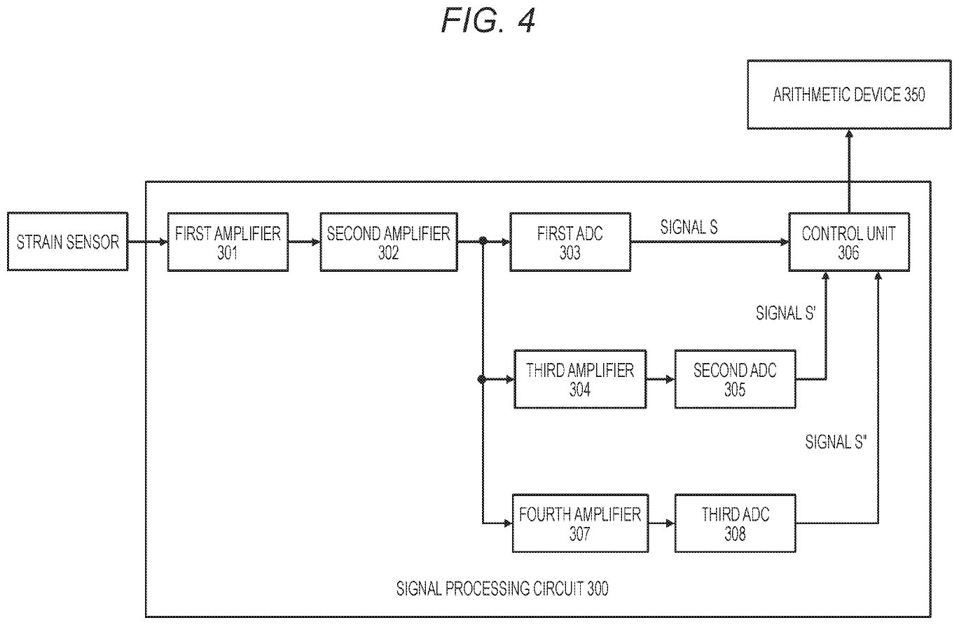

FIG. 4 illustrates a modification of the signal processing circuit 300. Note that the same configuration elements as those illustrated in FIG. 3 are given the same reference numerals.

The strain sensor in the figure corresponds to one of the strain sensors 121a and 121b, 122a and 122b, and 123a and 123b in the force sensor 100 illustrated in FIG. 1, for example. Basically, it is to be understood that the signal processing circuit 300 as illustrated in FIG. 4 is provided for each pair of strain sensors, and detection signals of the strain sensors are processed. Translational forces and torques applied to the plurality of axes are applied to each of the strain sensors 121a and 121b, 122a and 122b, and 123a and 123b in a complex manner, and therefore a detection signal of the strain sensors includes a plurality of components of the translational forces and torques.

When the detection signal of the strain sensor is input, the detection signal is amplified to achieve a necessary (or high) sensitivity that meets the purpose by two-stage amplification processing using the first amplifier 301 and the second amplifier 302. An output signal of the second amplifier 302 is branched into three paths having different amplification factors.

In the first path, the output signal of the second amplifier 302 is directly converted into a digital signal by a first AD converter (ADC) 303 and input to a control unit 306 at a subsequent stage as a high-sensitivity detection signal S. That is, in the one of the paths, the detection signal S corresponding to F_{z} to be detected with high sensitivity is created.

In the second path, the output signal of the second amplifier 302 is further amplified by a third amplifier 304, converted into a digital signal by a second AD converter 305, and input to a control unit 306 at a subsequent stage. Specifically, the third amplifier 304 is an attenuator that attenuates an input signal to 1/n (where n>1), and the input signal is converted into a digital signal by the second AD converter 305 and is then input to the control unit 306 as a low-sensitivity detection signal S′.

Moreover, in the third path, the output signal of the second amplifier 302 is further amplified by a fourth amplifier 305, converted into a digital signal by a third AD converter 308, and input to the control unit 306 at a subsequent stage. Specifically, the fourth amplifier 307 is an attenuator that attenuates an input signal to 1/n (where m>n), and the input signal is converted into a digital signal by the second AD converter 305 and is then input to the control unit 306 as a lower-sensitivity detection signal S″ than the above-described detection signal S′.

Then, the control unit 306 performs communication of digital data of the plurality of detection signals S, S′, and S″ having different sensitivities obtained from one strain sensor to the external arithmetic device (personal computer or robot control device) 350 and other types of digital processing.

According to the signal processing circuit 300 illustrated in FIG. 4, signals having different sensitivities at different rating levels can be simultaneously created by branching the detection signal of the strain sensor to generate the plurality of detection signals having different amplification factors.

According to the signal processing circuit 300 illustrated in FIG. 4, the number of detection signals having different amplification factors increases by one, so an effect of expanding the maximum measurable range or improving the resolution while keeping the maximum range constant can be expected, as compared with the configuration example illustrated in FIG. 3.

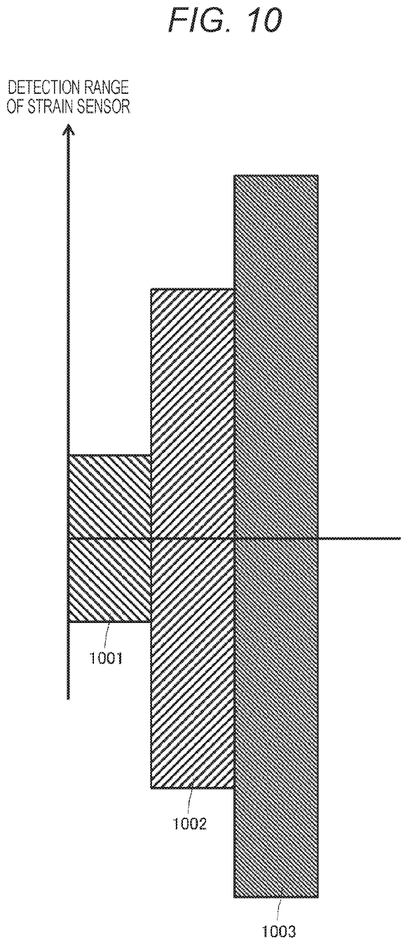

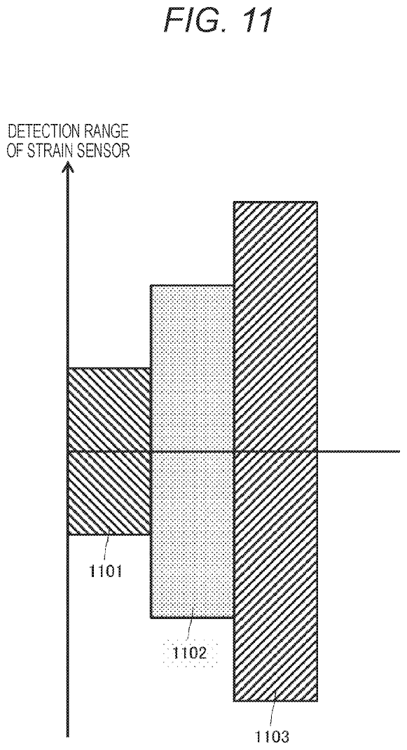

FIGS. 10 and 11 illustrate respective measurement ranges of the detection signal S output from the first AD converter 303, the detection signal S′ output from the second AD converter 305, and the detection signal S″ output from the third AD converter 306 in the signal processing circuit 300 illustrated in FIG. 4. FIG. 10 illustrates the measurement ranges of the detection signals S, S′, and S″ by reference numerals 1001, 1002, and 1003, respectively. The maximum measurable range 1003 is expanded with respect to the signal processing circuit 300 illustrated in FIG. 3. Meanwhile, FIG. 11 illustrates the measurement ranges of the detection signals S, S′, and S″ by reference numerals 1101, 1102, and 1103, respectively. The maximum measurable range 1103 is the same as that of the signal processing circuit 300 illustrated in FIG. 3 but the resolution is improved by the moderately attenuated detection signal S′.

The difference between the configuration example of the signal processing circuit 300 illustrated in FIG. 3 and the configuration example illustrated in FIG. 4 is that the amplifier is duplexed or tripled. Although illustration is omitted, there may be a configuration of the signal processing circuit 300 in which the amplifier is multiplexed four times or more.

Note that, in a case of multiplexing an amplifier, use of changing an offset of each amplifier and sharing the measurement range is conceivable in addition to the use of changing the amplification factor for each amplifier and creating a plurality of detection signals having different sensitivities as in the example illustrated in FIG. 4.

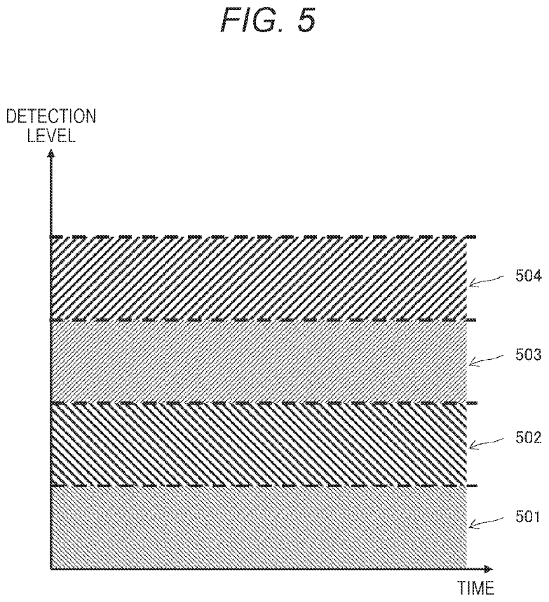

FIG. 5 illustrates a state in which a measurement range is shared by N-multiplexed amplifiers. In the figure, the vertical axis represents a detection level. A region illustrated by a reference numeral 501, of the measurement ranges, indicates a range that can be measured by the amplifier disposed on the first branch. Similarly, regions illustrated by reference numerals 502, 503, and 504 indicate ranges that can be measured by the amplifiers disposed on the second, third, and fourth branches, respectively.

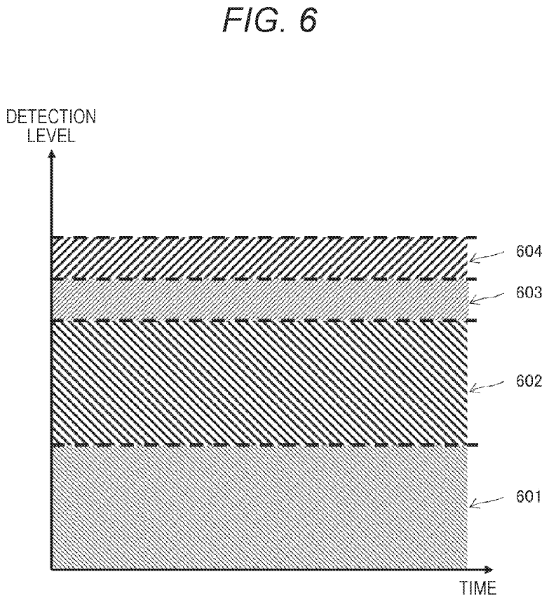

Furthermore, FIG. 6 illustrates another example in which a measurement range is shared by N-multiplexed amplifiers. In the figure, the vertical axis represents a detection level. A region illustrated by a reference numeral 601, of the measurement ranges, indicates a range that can be measured by the amplifier disposed on the first branch. Similarly, regions illustrated by reference numerals 602, 603, and 604 indicate ranges that can be measured by the amplifiers disposed on the second, third, and fourth branches, respectively. In the example illustrated in FIG. 5, the same attenuation factor is set to the multiplexed amplifiers, and therefore the widths of ranges shared by the amplifiers are uniform. In contrast, in the example illustrated in FIG. 6, the attenuation factors of the multiplexed amplifiers are varied, and therefore the widths of ranges shared by the amplifiers are not the same.

For example, the attenuation factor of the amplifier that shares a region that needs attention is made small to have a high sensitivity although the width of the range becomes narrow. On the contrary, the attenuation factor of the amplifier that shares a region that does not need attention is made large to have a low sensitivity, so that the width of the range can be made wide. In the example illustrated in FIG. 6, the regions illustrated by the reference numerals 601 and 602 are the regions that do not need attention. By making the attenuation factors of the amplifiers that share the regions large, the measurement range per amplifier is widened although the sensitivity becomes low. Meanwhile, the regions illustrated by the reference numerals 603 and 604 are the regions that need attention. By making the attenuation factors of the amplifiers that share the regions small, detection can be made with a high sensitivity although the measurement range per amplifier becomes narrow.

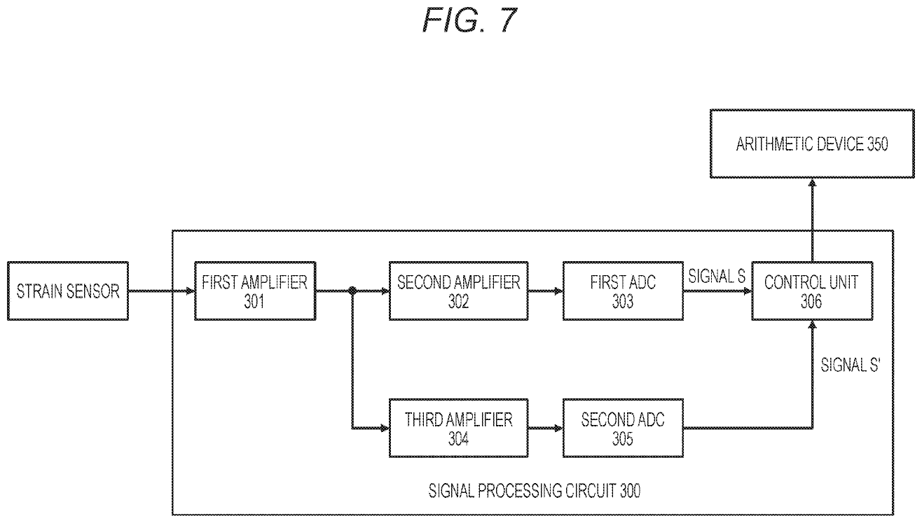

Furthermore, FIG. 7 illustrates another modification of the signal processing circuit 300. Note that the same configuration elements as those illustrated in FIG. 3 are given the same reference numerals. The signal processing circuit 300 illustrated in FIG. 7 is different from the configuration example illustrated in FIG. 3 in immediately branching after the first amplifier 301 and multiplexing the amplifiers.

The strain sensor in the figure corresponds to one of the strain sensors 121a and 121b, 122a and 122b, and 123a and 123b in the force sensor 100 illustrated in FIG. 1, for example. Basically, it is to be understood that the signal processing circuit 300 as illustrated in FIG. 7 is provided for each pair of strain sensors, and detection signals of the strain sensors are processed. Translational forces and torques applied to the plurality of axes are applied to each of the strain sensors 121a and 121b, 122a and 122b, and 123a and 123b in a complex manner, and therefore a detection signal of the strain sensors includes a plurality of components of the translational forces and torques.

The first amplifier 301 receives a detection signal of the strain sensor and amplifies the detection signal with low noise. Then, the output signal of the first amplifier 301 is branched into two paths having different amplification factors.

In one of the paths, as for the output signal of the first amplifier 301, the second amplifier 302 amplifies the detection signal after low-noise amplification with a predetermined amplification factor, and further appropriately performs processing such as offset adjustment as necessary. Then, the output signal is converted into a digital signal by the first AD converter (ADC) 303 as it is and input to the control unit 306 at a subsequent stage as the high-sensitivity detection signal S. That is, in the one of the paths, the detection signal S corresponding to F_{z} to be detected with high sensitivity is created.

Furthermore, in the other path, the output signal of the first amplifier 301 is further amplified by the third amplifier 304, converted into a digital signal by the second AD converter 305, and input to the control unit 306 at a subsequent stage. Specifically, the third amplifier 304 is an attenuator that attenuates an input signal to 1/n (where n>1), and the input signal is converted into a digital signal by the second AD converter 305 and is then input to the control unit 306 as a low-sensitivity detection signal S′. Furthermore, the third amplifier 304 further appropriately performs processing such as offset adjustment as necessary.

That is, in the other path, the detection signal S′ corresponding to the sensitivity of a signal other than F_{z} for which noise influence is to be reduced is created. For example, the third amplifier 304 weakens (or attenuates) the detection signal S such that the resolution becomes about ¼ of the maximum value of values that can be taken by the high-sensitivity detection signal S or breaking strength in which the strain generation body and the strain sensor do not break falls in the maximum range. Note that the third amplifier 304 may be a variable amplifier having a variable attenuation factor (1/n).

As described above, according to the technology disclosed in the present specification, a multi-axial force sensor capable of easily changing the ratio of the translational force to the torque can be implemented without changing the structure of the strain generation body. Furthermore, the rated measurement range of the force sensor can be changed without changing the structure of the strain generation body.

### SECOND EXAMPLE

In the first example, the detection signal of the strain sensor has been branched into the plurality of paths having different amplification factors, and the detection signal having the wide measurement range has been created through the path having the high amplification factor (for example, FIGS. 9 to 11). In contrast, the present example proposes a signal processing circuit capable of extending a measurement range while maintaining a high resolution.

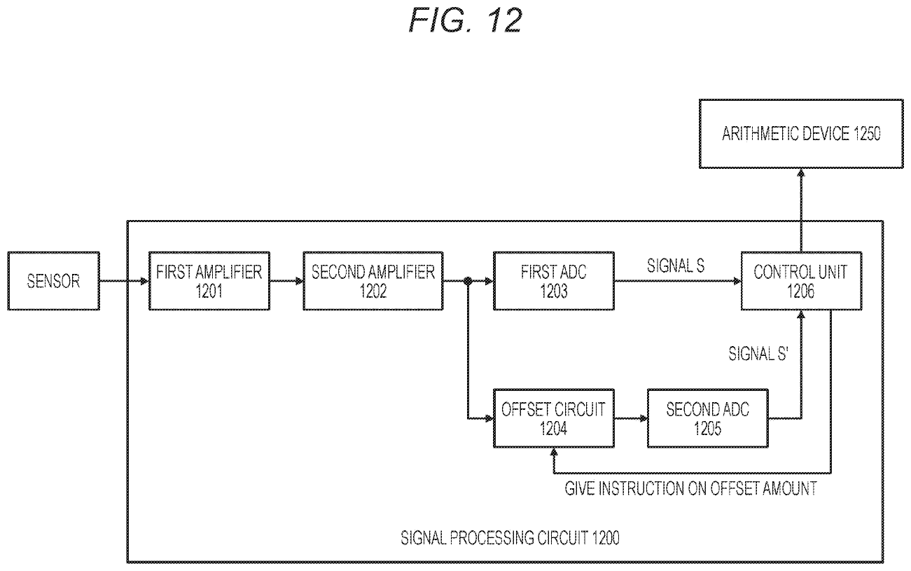

FIG. 12 illustrates a configuration example of a signal processing circuit 1200 according to a second example. The illustrated signal processing circuit 1200 is implemented in the form of an amplifier device connected to the force sensor 100 or a sensor such as a potentiometer, a communication unit that transmits an output signal of the sensor to an arithmetic device 1250 such as a personal computer or a robot control device, or the like.

A first amplifier 1201 receives a detection signal of the sensor and amplifies the detection signal with low noise. Furthermore, a second amplifier 1202 amplifies the detection signal after low-noise amplification with a predetermined amplification factor, and further appropriately performs processing such as offset adjustment as necessary. The input signal from the sensor is amplified to achieve a necessary (or high) sensitivity that meets the purpose by two-stage amplification processing using the first amplifier 1201 and the second amplifier 1202.

An output signal of the second amplifier 1202 is branched into two paths. In one of the paths, the output signal of the second amplifier 1202 is directly converted into a digital signal by a first AD converter (ADC) 1203 and input to a control unit 1206 at a subsequent stage as a high-sensitivity detection signal S. That is, in the one of the paths, the detection signal S corresponding to a high sensitivity is created although a measurement range is narrow.

Furthermore, in the other path, an offset amount of the output signal of the second amplifier 1202 is adjusted by an offset circuit 1204, then the output signal is converted into a digital signal by a second AD converter 1205, and input to the control unit 1206 at a subsequent stage as a high-sensitivity detection signal S′. The offset circuit 1204 has a circuit configuration that enables dynamic change in the offset amount of the input signal. In the signal processing circuit 1200, the control unit 1206 is configured to give an instruction on the offset amount to the offset circuit 1204 moment by moment. That is, in the other path, the detection signal S′ having the same resolution as that of the one path but having a measurement range that dynamically varies according to the offset amount given in instruction from the control unit 1206 is created.

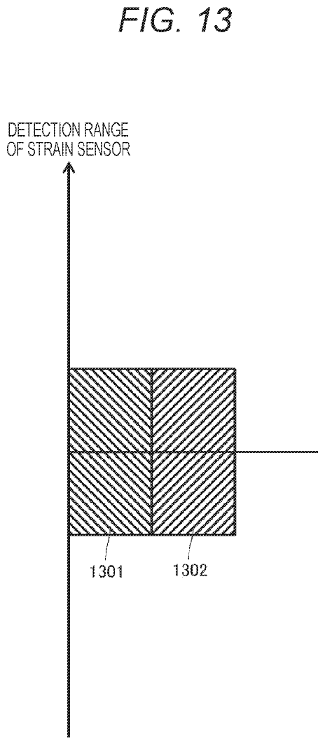

An operation example of the signal processing circuit 1200 will be described with reference to FIGS. 13 to 15.

FIG. 13 illustrates a measurement range 1301 of the detection signal S output from the first AD converter 1203, and a measurement range 1302 of the detection signal S′ output from the second AD converter 1205 in a case where the offset amount of the offset circuit 1204 is zero. In this case, the measurement range 1301 of the detection signal S and the measurement range 1302 of the detection signal S′ are exactly the same. Therefore, a high-resolution and narrow measurement range by only one system of the first amplifier 1201, the second amplifier 1202, and the first AD converter 1203 is a sensor detection range by the signal processing circuit 1200.

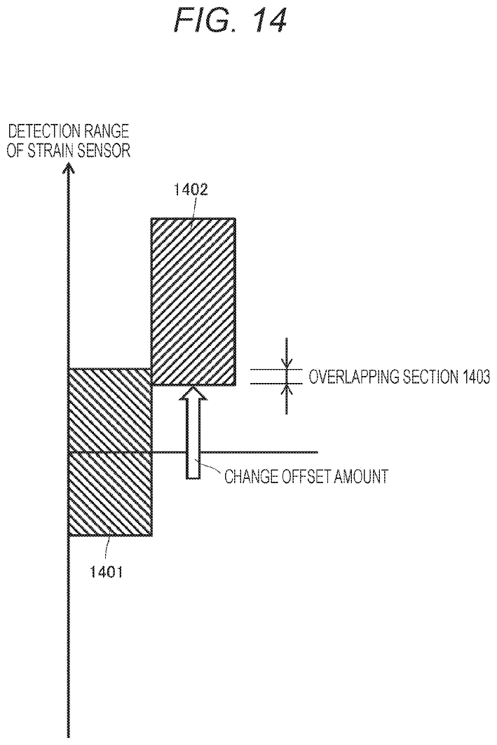

FIG. 14 illustrates a measurement range 1401 of the detection signal S output from the first AD converter 1203, and a measurement range 1402 of the detection signal S′ output from the second AD converter 1205 in a case where the offset amount of the offset circuit 1204 is shifted upward. In this case, the measurement range 1401 of the detection signal S is constant. Furthermore, the measurement range 1402 of the detection signal S′ is shifted upward according to the offset amount of the offset circuit 1204 while maintaining the high resolution. Therefore, the sensor detection range by the signal processing circuit 1200 is extended to a wide range that is a combination of the measurement range 1401 of the detection signal S and the measurement range 1402 of the detection signal S′, and can maintain the high resolution even in the extended measurement range 1402.

In the signal processing circuit 1200, the control unit 1206 is configured to give an instruction on the offset amount to the offset circuit 1204 moment by moment. For example, when the detection level of one detection signal S rises and approaches an upper limit of the measurement range 1401, the control unit 1206 is only required to output an instruction to the offset circuit 1204 to shift the offset amount upward.

When periodic change of an output signal of a sensor is known, the control unit 1206 may predict variation of the detection signal S and predictively control the offset amount of the offset circuit 1204. Furthermore, the control unit 1206 may introduce machine learning and predictively control the offset amount of the offset circuit 1204.

The sensor detection range by the signal processing circuit 1200 is extended more upward as the offset amount given by the offset circuit 1204 is larger. Note that, when switching from the measurement range 1401 of the detection signal S to the measurement range 1402 of the detection signal S′ is discontinuous, values to be input to the control unit 1206 becomes indeterminate, and there is a risk of runaway. Therefore, it is desirable to provide an overlapping section 1403 where an upper end of the measurement range 1401 and a lower end of the measurement range 1402 overlap above a certain level to cause the input signal from the sensor to fall within at least one of the measurement ranges.

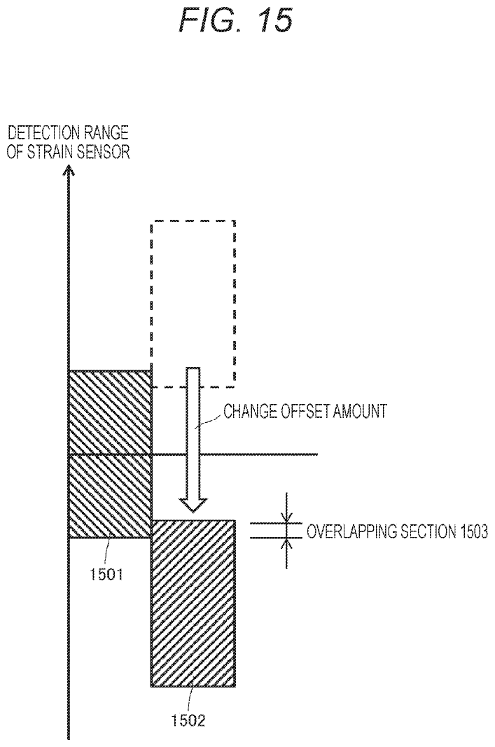

Furthermore, FIG. 15 illustrates a measurement range 1501 of the detection signal S output from the first AD converter 1203, and a measurement range 1502 of the detection signal S′ output from the second AD converter 1205 in a case where the offset amount of the offset circuit 1204 is shifted downward. The sensor detection range by the signal processing circuit 1200 in this case is a range of a combination of the measurement range 1501 of the detection signal S and the measurement range 1502 of the detection signal S′, and is extended downward according to the offset amount of the offset circuit 1204 and can maintain the high resolution over the entire range.

For example, when the detection level of one detection signal S rises and approaches a lower limit of the measurement range 1501, the control unit 1206 is only required to output an instruction to the offset circuit 1204 to shift the offset amount downward. The control unit 1206 may predict variation of the detection signal S and predictively control the offset amount of the offset circuit 1204. Furthermore, machine learning may be introduced to the control unit 1206 for predictive control. Furthermore, it is desirable to provide an overlapping section 1503 where a lower end of the measurement range 1501 and an upper end of the measurement range 1502 overlap above a certain level (same as above).

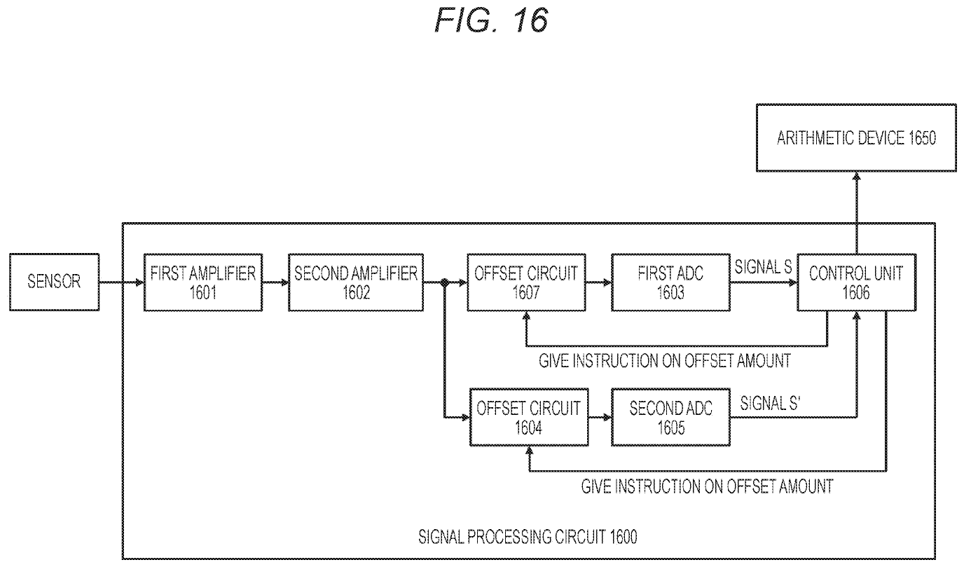

FIG. 16 illustrates another configuration example of a signal processing circuit 1600 according to the second example. The illustrated signal processing circuit 1600 is implemented in the form of an amplifier device connected to a sensor, a communication unit that transmits an output signal of the sensor to an arithmetic device 1650 such as a personal computer or a robot control device, or the like.

A first amplifier 1601 receives a detection signal of the sensor and amplifies the detection signal with low noise. Furthermore, a second amplifier 1602 amplifies the detection signal after low-noise amplification with a predetermined amplification factor, and further appropriately performs processing such as offset adjustment as necessary. The input signal from the sensor is amplified to achieve a necessary (or high) sensitivity that meets the purpose by two-stage amplification processing using the first amplifier 1601 and the second amplifier 1602.

An output signal of the second amplifier 1602 is branched into two paths having different offset amounts. In one of the paths, the offset amount of the output signal of the second amplifier 1602 is adjusted by an offset circuit 1607, then the output signal is converted into a digital signal by a first AD converter (ADC) 1603, and input to a control unit 1606 at a subsequent stage as a high-sensitivity detection signal S. Furthermore, in the other path, the offset amount of the output signal of the second amplifier 1602 is adjusted by an offset circuit 1604, then the output signal is converted into a digital signal by a second AD converter 1605, and input to the control unit 1606 at a subsequent stage as a high-sensitivity detection signal S′.

Both of the offset circuits 1604 and 1607 have a circuit configuration that enables dynamic change in the offset amount of the input signal. In the signal processing circuit 1600, the control unit 1606 can control the offset amounts of the offset circuits 1604 and 1607 independently of each other. Therefore, in each of the paths, the detection signals S and S′ having the same resolution and individually set offset amounts are created. Each path has a measurement range according to the offset amount set in each of the offset circuits 1604 and 1607.

The control unit 1606 adjusts the offset amount of each path so that the input signal from the sensor falls within the measurement range of at least one of the paths. Furthermore, there is a risk of runaway if the offset amount changes during AD conversion processing. Therefore, it is desirable to adjust the offset amount on the path side where the AD conversion processing is not being performed while keeping the offset amount fixed in the path during the AD conversion processing.

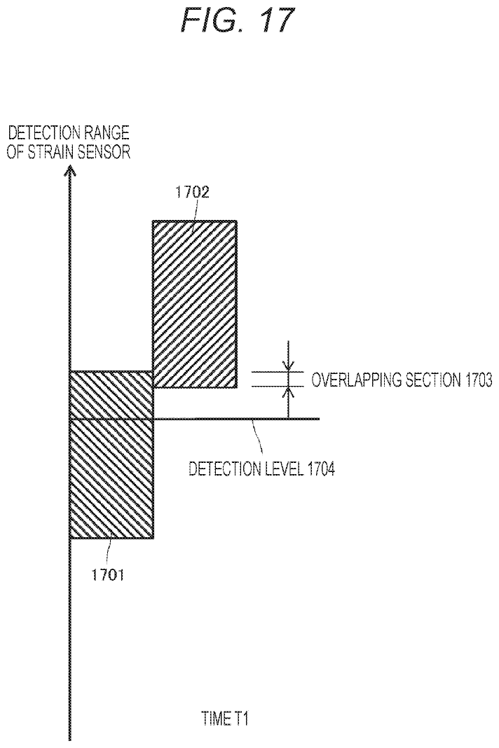

An operation example of the signal processing circuit 1600 will be described with reference to FIGS. 17 to 20.

FIG. 17 illustrates a measurement range 1701 of the detection signal S and a measurement range 1702 of the detection signal S′ at certain time T1. The sensor detection range by the signal processing circuit 1600 in this case is a range of a combination of the measurement range 1701 of the detection signal S and the measurement range 1702 of the detection signal S′.

The input signal from the sensor at this time T1 is a detection level illustrated by a reference numeral 1704 in the figure. That is, the detection level 1704 is in the upper half of the measurement range 1701 of the path currently undergoing the AD conversion processing, and it is predicted that the detection level 1704 will exceed an upper end of the measurement range 1701 in the near future. Dynamically changing the offset amount in the path during the AD conversion processing should be avoided, and the measurement range 1701 is fixed. Therefore, in the other path where the AD conversion processing is not being performed, the offset amount is adjusted to shift the measurement range 1702 upward above the upper end of the measurement range 1701 of the one path, so that the detection range of the signal processing circuit 1600 is extended upward to prepare for a situation where the detection level 1704 deviates from the measurement range 1701. Note that the offset amounts of the offset circuits 1604 and 1607 are adjusted to form an overlapping section 1703 where the upper end of the measurement range 1701 and a lower end of the measurement range 1702 overlap above a certain level.

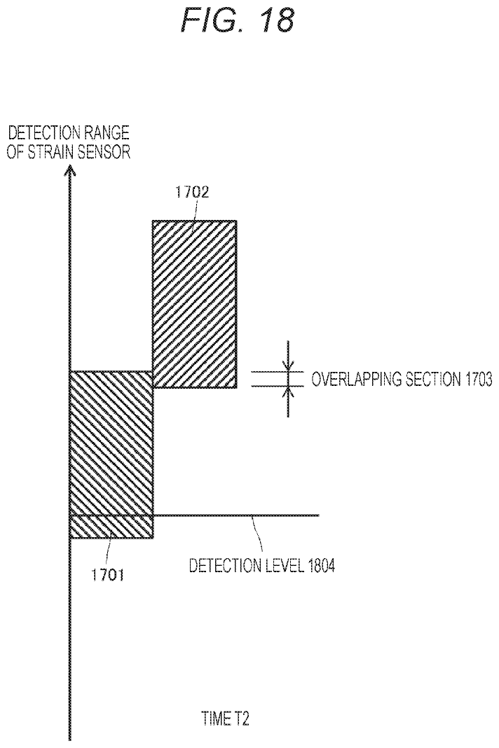

FIG. 18 illustrates a state in which the input signal from the sensor is lowered to the detection level illustrated by a reference numeral 1804 in FIG. 18 at subsequent time T2 (where T2>T1). The detection level 1804 is in the lower half of the measurement range 1701 of the path currently undergoing the AD conversion processing, and it is predicted that the detection level 1804 will exceed a lower end of the measurement range 1701 in the near future. Dynamically changing the offset amount in the path during the AD conversion processing should be avoided, and the measurement range 1701 is fixed. Therefore, in the other path where the AD conversion processing is not being performed, the offset amount is adjusted to shift the measurement range 1702 downward below the lower end of the measurement range 1701, so that the detection range of the signal processing circuit 1600 should be extended downward to prepare for a situation where the detection level 1804 deviates from the measurement range 1701.

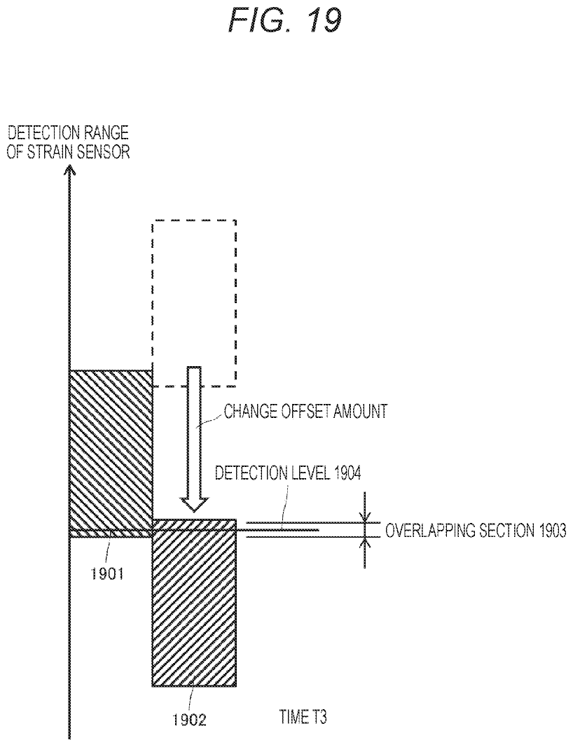

FIG. 19 illustrates a state in which, at further subsequent time T3 (where T3>T2), in the other path where the AD conversion processing is not being performed, the offset amount is adjusted to shift the measurement range 1702 downward below the lower end of the measurement range 1801 of the one path, so that the detection range of the signal processing circuit 1600 is extended downward to prepare for a situation where the detection level 1904 deviates from the measurement range 1801. Note that the offset amounts of the offset circuits 1604 and 1607 are adjusted to form an overlapping section 1903 where a lower end of a measurement range 1901 and an upper end of a measurement range 1902 overlap above a certain level.

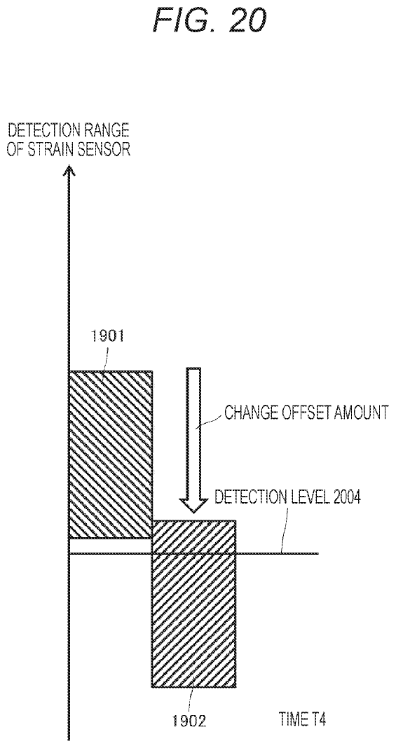

FIG. 20 illustrates a state in which, at further subsequent time T4 (where T4>T3), a detection level 2004 of the input signal from the sensor falls below the lower end of the measurement range 1901. Since the detection level 2004 falls within the measurement range 1902 of the other path, the processing is switched to the AD conversion processing by the other path (that is, the second AD converter 1605), and the detection signal S′ after the AD conversion is input to the control unit 1606 at a subsequent stage.

Note that, at the time T4, since the AD conversion processing is performed in the measurement range 1902 in the other path, the control unit 1606 fixes the offset amount of the offset circuit 1604. Furthermore, since the AD conversion processing is not performed in the one path, the control unit 1606 can adjust the offset amount of the offset circuit 1607 to shift the measurement range 1901.

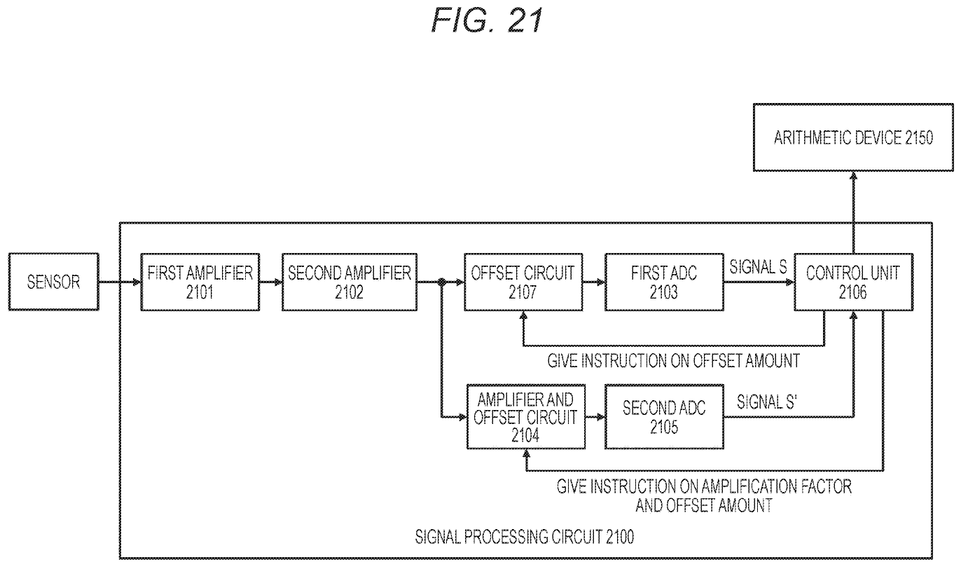

FIG. 21 illustrates still another configuration example of a signal processing circuit 2100 according to the second example.

A first amplifier 2101 receives a detection signal of the sensor and amplifies the detection signal with low noise. Furthermore, a second amplifier 2102 amplifies the detection signal after low-noise amplification with a predetermined amplification factor, and further appropriately performs processing such as offset adjustment as necessary. The input signal from the sensor is amplified to achieve a necessary (or high) sensitivity that meets the purpose by two-stage amplification processing using the first amplifier 2101 and the second amplifier 2102.

An output signal of the second amplifier 2102 is branched into two paths having different offset amounts. In one of the paths, the offset amount of the output signal of the second amplifier 2102 is adjusted by an offset circuit 2107, then the output signal is converted into a digital signal by a first AD converter (ADC) 2103, and input to a control unit 2106 at a subsequent stage as a high-sensitivity detection signal S. Furthermore, in the other path, the output signal of the second amplifier 2102 is amplified and the offset amount is adjusted by an amplifier and an offset circuit 2104, is then converted into a digital signal by a second AD converter 2105, and input to the control unit 2106 at a subsequent stage as a high-sensitivity detection signal S′.

The offset circuit 2107 has a circuit configuration that enables dynamic change in the offset amount of the input signal. Furthermore, the amplifier and the offset circuit 2104 have a circuit configuration to amplify (or attenuate) the input signal and enable dynamic change in the offset amount. In the signal processing circuit 2100, the control unit 2106 can control the offset amount of the offset circuit 2107 and the amplification factor and the offset amount of the amplifier and the offset circuit 2104 independently of one another. Therefore, in the one path, the detection signal S having the individually set offset amount is created, and in the other path, the detection signal S′ having a resolution according to the amplification factor and the individually set offset amount is created. Then, each path has the measurement range according to the offset amount and the amplification factor of the amplifier.

The control unit 2106 adjusts the offset amount of each path so that the input signal from the sensor falls within the measurement range of at least one of the paths. Furthermore, there is a risk of runaway if the offset amount changes during AD conversion processing. Therefore, the offset amount in the path during the AD conversion is kept fixed, and the offset amount is adjusted on the path side where the AD conversion processing is not being performed.

For example, during the period in which the first AD converter 2103 performs AD conversion processing for the input signal from the sensor, the control unit 2106 predicts variation of the detection level of the input signal from the sensor, and adjusts the offset amount and the amplification factor according to a desired resolution, of the amplifier and the offset circuit 2104 (increases the amplification factor to measure the signal with a high resolution while suppressing the amplification factor to reduce the influence of noise) while keeping the offset amount of the offset circuit 2107 fixed. Furthermore, during the period in which the second AD converter 2105 performs AD conversion for the input signal from the sensor, the control unit 2106 fixes the amplification factor and the offset amount of the amplifier and the offset circuit 2104, predicts the variation of the detection level of the input signal from the sensor, and adjusts the offset amount of the offset circuit 2107.

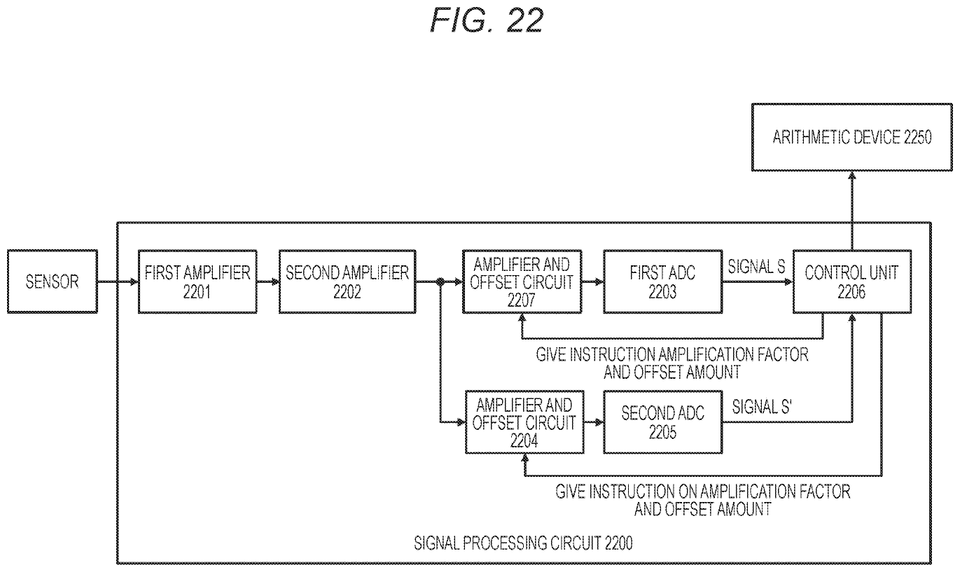

FIG. 22 illustrates still another configuration example of a signal processing circuit 2200 according to the second example.

A first amplifier 2201 receives a detection signal of the sensor and amplifies the detection signal with low noise. Furthermore, a second amplifier 2202 amplifies the detection signal after low-noise amplification with a predetermined amplification factor, and further appropriately performs processing such as offset adjustment as necessary. The input signal from the sensor is amplified to achieve a necessary (or high) sensitivity that meets the purpose by two-stage amplification processing using the first amplifier 2201 and the second amplifier 2202.

An output signal of the second amplifier 2202 is branched into two paths having different offset amounts. In one of the paths, the output signal of the second amplifier 2202 is amplified, the offset amount is adjusted by an amplifier and an offset circuit 2207, and the output signal is then input to a control unit 2206 at a subsequent stage as a sensitivity-adjusted detection signal S. Furthermore, in the other path, the output signal of the second amplifier 2202 is amplified and the offset amount is adjusted by an amplifier and an offset circuit 2204, is then converted into a digital signal by a second AD converter 2205, and input to the control unit 2206 at a subsequent stage as a sensitivity-adjusted detection signal S′.

All of the amplifier and the offset circuits 2207 and 2204 have a circuit configuration to amplify (or attenuate) the input signal and enable dynamic change in the offset amount. In the signal processing circuit 2200, the control unit 2206 can control the amplification factor and the offset amounts of the amplifier and both the offset circuits 2207 and 2204 independently of one another. Therefore, in the one path, the detection signal S having the individually set sensitivity and offset amount is created, and in the other path, the detection signal S′ having the individually set sensitivity and offset amount is created. Then, each path has the measurement range according to the offset amount and the amplification factor of the amplifier.

The control unit 2206 adjusts the offset amount of each path so that the input signal from the sensor falls within the measurement range of at least one of the paths. Furthermore, there is a risk of runaway if the offset amount changes during AD conversion processing. Therefore, the amplification factor and offset amount in the path during the AD conversion processing are kept fixed, and the amplification factor and offset amount are adjusted on the path side where the AD conversion processing is not being performed.

For example, during the period in which the first AD converter 2203 performs AD conversion processing for the input signal from the sensor, the control unit 2206 predicts variation of the detection level of the input signal from the sensor, and adjusts the offset amount and the amplification factor according to a desired resolution, of the amplifier and the offset circuit 2204 (increases the amplification factor to measure the signal with a high resolution while suppressing the amplification factor to reduce the influence of noise) while keeping the amplification factor and the offset amount of the amplifier and the offset circuit 2207 fixed. Furthermore, during the period in which the second AD converter 2205 performs AD conversion for the input signal from the sensor, the control unit 2206 predicts the variation of the detection level of the input signal from the sensor, and adjusts the offset amount and the amplification factor according to the desired resolution, of the amplifier and the offset circuit 2207, while fixing the amplification factor and the offset amount of the amplifier and the offset circuit 2204.

### INDUSTRIAL APPLICABILITY

The technology disclosed in the present specification has been described in detail with reference to the specific embodiments. However, it is obvious that those skilled in the art can make modifications and substitutions of the embodiments without departing from the gist of the technology disclosed in the present specification.

The application target of the technology disclosed in the present specification is not limited to a specific strain generation body structure, and can be applied to, for example, a uniaxial load cell, a triaxial force sensor, a six-axis force sensor, and the like. Furthermore, the force sensor to which the technology disclosed in the present specification is applied can cope with the wide range of change in the ratio of the translational force to the torque, thereby constituting a robot arm capable of more versatilely working without being replaced for each use.

In short, the technology disclosed in the present specification has been described in the form of examples, and the contents of description of the present specification should not be restrictively construed. To judge the gist of the technology disclosed in the present specification, the scope of claims should be taken into consideration.

Note that the technology disclosed in the present specification may have the following configurations.

(1) A force detection device including: a signal processing unit configured to branch a detection signal of a sensor attached to a strain generation body, and generate a plurality of detection signals having different sensitivities.

(2) The force detection device according to (1), further including:

a first amplification unit configured to amplify the detection signal of the sensor to match a first sensitivity;

a first AD conversion unit configured to convert a signal of the first sensitivity output from the first amplification unit into a digital signal;

a second amplification unit branching from the output of the first amplification unit, and configured to attenuate the signal of the first sensitivity, and output a signal of a second sensitivity lower than the first sensitivity; and

a second AD conversion unit configured to convert the signal of the second sensitivity output from the second amplification unit into a digital signal.

(3) The force detection device according to (2), in which

the first amplification unit includes a low-noise amplifier that amplifies the detection signal of the sensor with low noise and an amplifier that amplifies, with a predetermined amplification factor, or adjusts an offset of a signal output from the low-noise amplifier.

(4) The force detection device according to (2) or (3), in which

the second amplification unit attenuates the signal of the first sensitivity such that a resolution becomes about 1/n of a maximum value of available values of the signal of the first sensitivity (where n>1) or breaking strength in which the strain generation body and the sensor do not break falls in a maximum range.

(5) The force detection device according to any one of (2) to (4), further including:

a third amplification unit branching from the output of the first amplification unit, and configured to attenuate the signal of the first sensitivity with an attenuation factor different from the second amplifier, and output a signal of a third sensitivity; and

a third AD conversion unit configured to convert the signal of the third sensitivity output from the third amplification unit into a digital signal.

(6) The force detection device according to any one of (2) to (5), further including:

a control unit configured to process a signal after digital conversion.

(7) The force detection device according to (6), in which

the control unit digitally communicates with an external arithmetic device.

(8) The force detection device according to any one of (1) to (7), in which

the sensor is a strain gauge or a deformation detection sensor of one of piezoelectric type, magnetic type, optical type, and capacitance type.

(9) A force detection device including:

a strain generation body;

a plurality of sensors attached to the strain generation body; and

a signal processing unit configured to branch at least one of detection signals of the plurality of sensors, and generate a plurality of signals having different sensitivities.

(10) The force detection device according to (9), in which

the signal processing unit performs communication of the plurality of signals after digital conversion with an external arithmetic device.

(11) The force detection device according to (9) or (10), further including:

an arithmetic unit configured to calculate a force or a torque to act on the strain generation body, using the plurality of signals having different sensitivities.

(12) The force detection device according to (11), in which

the arithmetic unit partially uses a signal of a second sensitivity lower than a first sensitivity when any of the plurality of signals of the first sensitivity reaches an upper limit.

(13) A force detection method including: a signal processing step of branching a detection signal of a sensor attached to a strain generation body, and generating a plurality of detection signals having different sensitivities.

(14) A robot device including:

an end effector;

a force sensor attached to a proximal end side of the end effector; and

a signal processing unit configured to process a detection signal of the force sensor, in which

the force sensor includes a strain generation body and a sensor that detects deformation of the strain generation body, and

the signal processing unit branches the detection signal of the sensor, and generates a plurality of detection signals having different sensitivities.

(15) The robot device according to (14), in which

the end effector includes a medical instrument.

(21) A signal processing device including: a signal processing unit configured to branch a detection signal of a sensor into a plurality of paths, and perform different preprocessing before AD conversion for each of the paths to generate a plurality of detection signals.

(22) The signal processing device according to (21), in which

a first path for performing AD conversion of a signal of a first sensitivity, the signal being obtained by amplifying the detection signal of the sensor to match the first sensitivity, and a second path for attenuating the signal of the first sensitivity and performing AD conversion of a signal of a second sensitivity lower than the first sensitivity, are included, and the plurality of detection signals having different sensitivities is generated.

(23) The signal processing device according to (22), in which

the sensor is a sensor attached to a strain generation body, and

in the second path, the signal of the first sensitivity is attenuated such that a resolution becomes about 1/n of a maximum value of available values of the signal of the first sensitivity (where n>1) or breaking strength in which the strain generation body and the sensor do not break falls in a maximum range.

(24) The signal processing device according to (22), in which

a third path for attenuating the signal of the first sensitivity and performing AD conversion of a signal of a third sensitivity lower than the first sensitivity and different from the second sensitivity, is further included.

(25) The signal processing device according to (21), in which

a first path for attenuating a signal of a first sensitivity, the signal of the first sensitivity being obtained by amplifying the detection signal of the sensor to match the first sensitivity, and performing AD conversion of a signal of a second sensitivity lower than the first sensitivity, and a second path for attenuating the signal of the first sensitivity and performing AD conversion of a signal of a third sensitivity lower than the first sensitivity and different from the second sensitivity, are included, and the plurality of detection signals having different sensitivities is generated.

(26) The signal processing device according to (21), in which

a path for changing an offset of a signal of a first sensitivity, the signal being obtained by amplifying the detection signal of the sensor to match the first sensitivity, and performing AD conversion is included.

(27) The signal processing device according to (21), in which

a first path for performing AD conversion of a signal of a first sensitivity, the signal being obtained by amplifying the detection signal of the sensor to match the first sensitivity, and a second path for changing an offset of the signal of the first sensitivity and performing AD conversion are included.

(28) The signal processing device according to (21), in which

a first path for changing an offset of a signal of a first sensitivity, the signal being obtained by amplifying the detection signal of the sensor to match the first sensitivity, and performing AD conversion, and a second path for setting the signal of the first sensitivity to an offset different from the offset of the first path and performing AD conversion are included

(29) The signal processing device according to (28), in which

the signal of the first sensitivity is attenuated or amplified to a signal of a sensitivity different from the first sensitivity in the second path.

(30) The signal processing device according to (28), in which

the signal of the first sensitivity is attenuated or amplified to a signal of a sensitivity different from the first sensitivity in each of the first and second paths.

(31) The signal processing device according to any one of (22) to (30), further including:

a first amplification unit including a low-noise amplifier that amplifies the detection signal of the sensor with low noise and an amplifier that amplifies, with a predetermined amplification factor, or adjusts an offset of a signal output from the low-noise amplifier, and configured to generate the signal of the first sensitivity from the detection signal of the sensor.

(32) The signal processing device according to any one of (22) to (31), further including:

a control unit configured to process a signal after AD conversion in each of the paths.

(33) The signal processing device according to (22), in which

the control unit digitally communicates with an external arithmetic device.

(34) A signal processing method including: a signal processing step of branching a detection signal of a sensor into a plurality of paths, and performing different preprocessing before AD conversion for each of the paths to generate a plurality of detection signals.

(35) A force detection device including: a signal processing unit configured to branch a detection signal of a sensor attached to a strain generation body into a plurality of paths, and perform different preprocessing before AD conversion for each of the paths to generate a plurality of detection signals.

(36) The force detection device according to (35), in which

the sensor is a strain gauge or a deformation detection sensor of any of piezoelectric type, magnetic type, optical type, and capacitance type.

(37) The force detection device according to (35) or (36), in which

the signal processing unit performs communication of the plurality of signals after digital conversion with an external arithmetic device.

(38) The force detection device according to (35) or (36), further including:

an arithmetic unit configured to calculate a force or a torque to act on the strain generation body, using the plurality of signals having different sensitivities.

(38-1) The force detection device according to (38), in which

the arithmetic unit partially uses a signal of a second sensitivity lower than a first sensitivity when any of the plurality of signals of the first sensitivity reaches an upper limit.

(39) A robot device including:

an end effector;

a force sensor attached to a proximal end side of the end effector; and

a signal processing unit configured to process a detection signal of the force sensor, in which

the force sensor includes a strain generation body and a sensor that detects deformation of the strain generation body, and

the signal processing unit branches the detection signal of the sensor, and performs different preprocessing before AD conversion for each path to generate a plurality of detection signals.

(40) The robot device according to (39), in which

the end effector includes a medical instrument.

### REFERENCE SIGNS LIST

100 Force sensor
110 Strain generation body
111, 112, 113 Support
114 Top plate
115 Bottom plate
121, 122, 123 Strain sensor
200 Forceps
201 Force sensor
202 Drive unit
300 Signal processing circuit
301 First amplifier
302 Second amplifier
303 First AD converter
304 Third amplifier
305 Second AD converter
306 Control unit
350 Arithmetic device

1. A signal processing device comprising: a signal processing unit configured to branch a detection signal of a sensor into a plurality of paths, and perform different preprocessing before AD conversion for each of the paths to generate a plurality of detection signals.

2. The signal processing device according to claim 1, wherein
a first path for performing AD conversion of a signal of a first sensitivity, the signal being obtained by amplifying the detection signal of the sensor to match the first sensitivity, and a second path for attenuating the signal of the first sensitivity and performing AD conversion of a signal of a second sensitivity lower than the first sensitivity, are included, and the plurality of detection signals having different sensitivities is generated.

3. The signal processing device according to claim 2, wherein
the sensor is a sensor attached to a strain generation body, and
in the second path, the signal of the first sensitivity is attenuated such that a resolution becomes about 1/n of a maximum value of available values of the signal of the first sensitivity (where n>1) or breaking strength in which the strain generation body and the sensor do not break falls in a maximum range.

4. The signal processing device according to claim 2, wherein
a third path for attenuating the signal of the first sensitivity and performing AD conversion of a signal of a third sensitivity lower than the first sensitivity and different from the second sensitivity, is further included.

5. The signal processing device according to claim 1, wherein
a first path for attenuating a signal of a first sensitivity, the signal of the first sensitivity being obtained by amplifying the detection signal of the sensor to match the first sensitivity, and performing AD conversion of a signal of a second sensitivity lower than the first sensitivity, and a second path for attenuating the signal of the first sensitivity and performing AD conversion of a signal of a third sensitivity lower than the first sensitivity and different from the second sensitivity, are included, and the plurality of detection signals having different sensitivities is generated.

6. The signal processing device according to claim 1, wherein
a path for changing an offset of a signal of a first sensitivity, the signal being obtained by amplifying the detection signal of the sensor to match the first sensitivity, and performing AD conversion is included.

7. The signal processing device according to claim 1, wherein
a first path for performing AD conversion of a signal of a first sensitivity, the signal being obtained by amplifying the detection signal of the sensor to match the first sensitivity, and a second path for changing an offset of the signal of the first sensitivity and performing AD conversion are included.

8. The signal processing device according to claim 1, wherein
a first path for changing an offset of a signal of a first sensitivity, the signal being obtained by amplifying the detection signal of the sensor to match the first sensitivity, and performing AD conversion, and a second path for setting the signal of the first sensitivity to an offset different from the offset of the first path and performing AD conversion are included.

9. The signal processing device according to claim 8, wherein
the signal of the first sensitivity is attenuated or amplified to a signal of a sensitivity different from the first sensitivity in the second path.

10. The signal processing device according to claim 8, wherein
the signal of the first sensitivity is attenuated or amplified to a signal of a sensitivity different from the first sensitivity in each of the first and second paths.

11. The signal processing device according to claim 2, further comprising:
a first amplification unit including a low-noise amplifier that amplifies the detection signal of the sensor with low noise and an amplifier that amplifies, with a predetermined amplification factor, or adjusts an offset of a signal output from the low-noise amplifier, and configured to generate the signal of the first sensitivity from the detection signal of the sensor.

12. The signal processing device according to claim 2, further comprising:
a control unit configured to process a signal after AD conversion in each of the paths.

13. The signal processing device according to claim 12, wherein
the control unit digitally communicates with an external arithmetic device.

14. A signal processing method comprising: a signal processing step of branching a detection signal of a sensor into a plurality of paths, and performing different preprocessing before AD conversion for each of the paths to generate a plurality of detection signals.

15. A force detection device comprising: a signal processing unit configured to branch a detection signal of a sensor attached to a strain generation body into a plurality of paths, and perform different preprocessing before AD conversion for each of the paths to generate a plurality of detection signals.

16. The force detection device according to claim 15, wherein
the sensor is a strain gauge or a deformation detection sensor of any of piezoelectric type, magnetic type, optical type, and capacitance type.

17. The force detection device according to claim 15, further comprising:
an arithmetic unit configured to calculate a force or a torque to act on the strain generation body, using the plurality of signals having different sensitivities.

18. The force detection device according to claim 17, wherein
the arithmetic unit partially uses a signal of a second sensitivity lower than a first sensitivity when one of the plurality of signals of the first sensitivity reaches an upper limit.

19. A robot device comprising:
an end effector;
a force sensor attached to a proximal end side of the end effector; and
a signal processing unit configured to process a detection signal of the force sensor, wherein
the force sensor includes a strain generation body and a sensor that detects deformation of the strain generation body, and
the signal processing unit branches the detection signal of the sensor, and performs different preprocessing before AD conversion for each path to generate a plurality of detection signals.

20. The robot device according to claim 19, wherein
the end effector includes a medical instrument.
